(19) United States
(12) Patent Application Publication
(10) Pub. No.: US 2006/0051332 A1
(43) Pub. Date: Mar. 9, 2006

(54) Generation of histocompatible tissues using nuclear transplantation

(75) Inventor: Robert Lanza, Clinton, MA (US)

Correspondence Address: FISH & NEAVE IP GROUP;ROPES & GRAY LLP, 1251 AVENUE OF THE AMERICAS FL C3, NEW YORK, NY, 10020-1105, US

(73) Assignee: Advanced Cell Technology, Inc., Worcester, MA (US)

(21) Appl. No.: 10/515,705

(22) PCT Filed: May 23, 2003
(86) PCT No.: PCT/US03/16424

§ 371 Date: Sep. 28, 2005

Related U.S. Application Data
(60) Provisional application No. 60/382,386, filed on May 23, 2002.

(51) Int. Cl. A61K 48/00; C12N 5/08

(52) U.S. Cl. 424/93.21; 435/366

(57) ABSTRACT

Tissues produced by culture of cells produced by nuclear transfer on a matrix derived from nuclear transfer embryos or embryos and pluripotent cells provided by other methods are provided. These tissues are useful for cell therapy.

### FIELD OF THE INVENTION

This invention relates to the use of cells and tissues produced by nuclear transplantation cloning methods for transplantation and cell therapy.

### BACKGROUND OF THE INVENTION

Nuclear transplantation (therapeutic cloning) could theoretically provide a limitless source of cells for regenerative therapy. Although the cloned cells would carry the nuclear genome of the patient, the presence of mitochondria inherited from the recipient oocyte raises questions about the histocompatibility of the resulting cells. In this study, we created bioengineered tissues from cardiac, skeletal muscle, and renal cells cloned from adult bovine fibroblasts. Long-term viability was demonstrated after transplantation of the grafts back into the nuclear donor animals. Reverse transcription-PCR (RT-PCR) and western blot analysis confirmed the expression of specific mRNA and proteins in the retrieved tissues despite expressing a different mitochondrial DNA (mtDNA) haplotype. In addition to creating skeletal muscle and cardiac ‘patches,’ nuclear transplantation was used to generate functioning renal units that produced urinelike fluid and demonstrated unidirectional secretion and concentration of urea nitrogen and creatinine. Examination of the explanted renal devices revealed formation of organized glomeruli- and tubule-like structures. Delayed-type hypersensitivity (DTH) testing in vivo and Elispot analysis in vitro suggested that there was no rejection response to the cloned cells. The ability to generate histocompatible cells using cloning techniques would overcome one of the major scientific challenges in transplantation medicine.

According to data from the Centers for Disease Control, as many as 3,000 Americans die every day from diseases that in the future may be treatable with embryonic stem (ES)-derived tissues’. In addition to generating functional replacement cells such as cardiomyocytes, neurons or insulin-producing B-cells, there is also the possibility that these cells could be used to reconstitute more complex tissues and organs, including blood vessels, myocardial “patches,” kidneys, and even entire hearts²⁻⁴. Somatic cell nuclear transfer (SCNT) has the potential to eliminate immune responses associated with the transplantation of these various tissues, and thus the requirement for immunosuppressive drugs and/or immunomodulatory protocols that carry the risk of a wide variety of serious and potentially life-threatening complication⁵.

### BRIEF DESCRIPTION OF THE FIGURES

FIG. 1. Retrieved muscle tissues: A. Cloned cardiac tissue retrieved shows a well-organized cellular orientation 6 weeks after implantation. H & E, reduced from 200×. B. Immunocytochemical analysis using troponin I antibodies identifies cardiac fibers within the implanted constructs 6 weeks after implantation. Reduced from 200×. C. Cardiac cell implant in control group shows fibrosis and necrotic debris in 6 weeks. H & E, reduced from 100×. D. Cloned skeletal muscle cell implants shows well-organized bundle formation. H & E, reduced from 40×. E. Retrieved skeletal cell implant with polymer fibers. H & E, reduced from 200×. F. Immunohistochemical analysis using sarcomeric tropomyosin antibodies identifies skeletal fibers within the implanted second-set constructs 12 weeks after implantation. Reduced from 40×. G. Retrieved cloned skeletal cell implants show spatially oriented muscle fiber 12 weeks after implantation. H & E, reduced from 100×. H. Retrieved control skeletal cell implant shows fibrosis with increased inflammatory reaction in 12 weeks. H & E, reduced from 40×. 1. Skeletal muscle cell implant in control group shows an increased number of inflammatory cells, fibrosis, and necrotic debris in 12 weeks. H & E, reduced from 100×. J. Immunocytochernical analysis using CD4 antibodies identifies CD4+ T cells within the implanted control cardiac construct 6 weeks after implantation. Reduced from 100×.

FIG. 2. RT-PCR and Western blot analyses. Semi-quantitative RT-PCR products indicate specific mRNA in the retrieved skeletal muscle tissue (A) and cardiac muscle tissue (B); the control group at 6 weeks, CL 6; the cloned group at 6 weeks, CO 12; the control group at 12 weeks, CL 12; the cloned group at 12 weeks. Western blot analysis of the implants confirmed the expression of specific proteins in the skeletal muscle tissues (A) and cardiac muscle tissues (B); the control group in 6 weeks, CL 6; the cloned group at 6 weeks, CO 12; the control group at 12 weeks, CL 12; the cloned group at 12 weeks.

FIG. 3. Tissue-engineered renal units. Illustration of renal unit (A) and units retrieved 3 months after implantation. B. Unseeded control. C. Seeded with allogeneic control cells. D. Seeded with cloned cells, showing the accumulation of urine-like fluid.

FIG. 4. Characterization of renal explants. A. Cloned cells stained positively with synaptopodin antibody (A) and AQP1 antibody (B). The allogeneic controls displayed a foreign body reaction with necrosis (C). Cloned explant shows organized glomeruli (D) and tubule (E)-like structures. H&E, reduced from 400×. Immunohistochemical analysis using factor VIII antibodies identifies vascular structure within D (F). Reduced from ×400. G. There was a clear unidirectional continuity between the mature glomeruli, their tubules, and the polycarbonate membrane.

FIG. 5. RT-PCR analyses (upper) confirming the transcription of AQP1, AQP2, Tamm-Horsfall protein and synaptopodin genes exclusively in the cloned group (CIs). Western blot analysis (lower) confirms high protein levels of AQP1 and AQP2 in the cloned group, whereas expression intensities of CD4 and CD8 were significantly higher in the control allogeneic group (Col&2).

FIG. 6. Elispot analyses of the frequencies of T cells that secrete IFN-gamma following primary and secondary stimulation with allogeneic renal cells, cloned renal cells, or nuclear donor fibroblasts. The presented wells are single representatives of the duplicate wells for each responder: stimulator combination.

FIG. 7. RT-PCR analyses (upper) confirming the transcription of AQP1, AQP2, Tamm-Horsfall protein and synaptopodin genes exclusively in the cloned group (Cls). Western blot analysis (lower) confirms high protein levels of AQP1 and AQP2 in the cloned group, whereas expression intensities of CD4 and CD8 were significantly higher in the control allogeneic group (Co1&2).

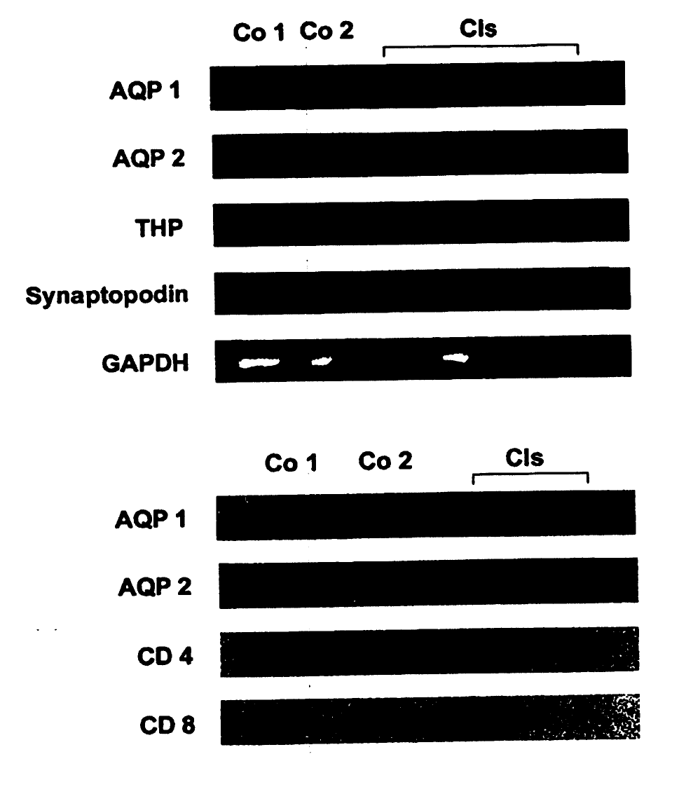

FIG. 8. Elispot analyses of the frequencies of T cells that secrete IFN-gamma following primary and secondary stimulation with allogeneic renal cells, cloned renal cells, or nuclear donor fibroblasts. The presented wells are single representatives of the duplicate wells for each responder.stimulator combination.

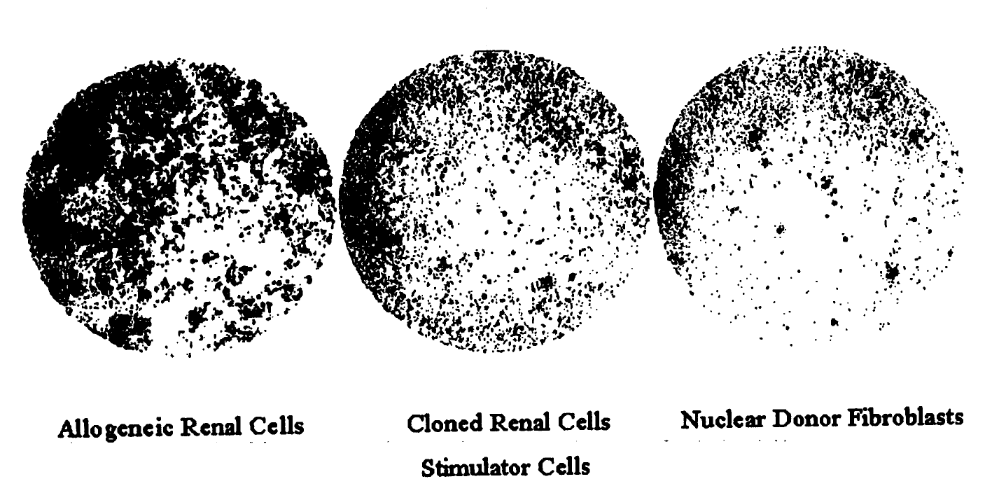

### SUMMARY OF THE INVENTION

Therefore, T is an object of the invention to provide cell and tissue transplantation therapies that use cells and tissues provided by nuclear transfer cloning methods.

More specifically, it is an object of the invention to provide cell and tissue transplantation therapies that utilize cells and tissues produced by nuclear transfer cloning methods and optionally in vitro tissue engineering, wherein such cells and tissues express allogenic or xenogenic mitochondrial DNA relative to the transplant recipient.

Even more specifically it is an object of the invention to treat human recipients in need of cell or tissue therapy using cells or tissues produced by nuclear transplantation of a human donor cell or human nuclear or chromosomal DNA, which is optionally transgenic, into a recipient oocyte, which is activated before, during and/or after nuclear transfer, resulting in a nuclear transfer embryo, the cells of which are used to derive specific cell types, e.g., ES cells and desired differentiated cell types, and which are then placed on a tissue matrix resulting in a three-dimensional tissue.

It is a specific object of the invention to obtain desired differentiated cell types derived from a nuclear transfer embryo, culture said cells in vitro or in vivo under conditions whereby such cells assemble (bioengineer) into a specific tissue type, e.g., kidney, heart, immune system, skeletal tissue, and transplant said bioengineered tissue into a recipient in need of cell or tissue therapy.

It is a more specific object of the invention to derive renal cells from a nuclear transfer embryo, culture said renal cells in vitro under conditions whereby said renal cells assemble into a tissue having morphological and functional characteristics of endogenous kidney, and transplanting said tissue into a recipient in need of renal cell or tissue therapy.

It is another more specific object of the invention to derive cardiac cells from a nuclear transfer embryo, culture said cardiac cells in vitro or in vivo under conditions whereby said cells assemble into a tissue having morphological and functional characteristics of endogenous cardiac tissue, and transplanting said cardiac tissue into a recipient in need of cardiac cell or tissue therapy.

It is another specific object of the invention to derive hepatic cells from a nuclear transfer embryo, culture said cells in vitro or in vivo under conditions whereby said cells assemble into a tissue having morphological and functional characteristics of endogenous hepatic tissue and implanting said hepatic tissue into a recipient in need of hepatic cells or tissue therapy.

It is still another object of the invention to derive pancreatic cells, e.g., islets from a nuclear transfer embryo, culture said cells in vitro under conditions whereby said cells assemble into a tissue having morphological and functional characteristics of endogenous pancreas and implanting said pancreatic tissue into a recipient in need of pancreatic cell or tissue therapy.

In some preferred embodiments, the engineered cells or tissues will express a transgene, e.g., one which encodes a therapeutic polypeptide.

In other preferred embodiments, the engineered cells or tissues will be administered as part of another therapy, e.g., in conjunction with other drugs for treating the condition that is to be alleviated by cell or tissue therapy. Such diseases and conditions include by way of example cancer, inflammatory disorders, autoimmune disorders, cell proliferation disorders, heart disease, pancreatic diseases such as type 1 and type 2 diabetes, kidney injury or disease, skeletal or bone injury or disease, immune cell deficiencies or dysfunction, lung injury or disease, reproductive organ dysfunction or disease, liver damage or disease, stomach injury or disease, intestinal dysfunction or disease, tracheal injury or disease, and the like.

As discussed in detail infra, it has been shown that bioengineered tissues derived from nuclear transfer embryos, particularly cardiac, skeletal and renal tissues, when implanted in vivo, do not elicit a rejection response and possess morphological and functional properties characteristic of endogenous skeletal, cardiac or renal tissue. This demonstrates that the expression of allogenic mitrochondrial DNA, or the nuclear transfer cloning process, did not result in the expression of antigenic epitopes by the cloned tissues which were problematic (elicit rejection) in the context of cell and tissue transplantation therapies.

Although the goal of “therapeutic” cloning is to generate replacement cells and tissues that are genetically identical with the donor, numerous studies have shown that animals produced by the SCNT technique inherit their mitochondria entirely or in part from the recipient oocyte and not the donor cell⁶⁻⁸. This raises the question of whether non-self mitochondrial proteins in cells could lead to immunogenicity after transplantation and defeat the main objective of the procedure. For instance, it has been demonstrated that mitochondrial peptides in mice are presented at the cell surface by non-classical MHC class I molecules in combination with beta-2-microglobulin⁹⁻¹⁰. It has also been shown that a single, nonsynonymous nucleotide substitution in the ND1 gene results in a novel peptide that can be recognized by specific cytotoxic T cells¹¹, A similar situation has been identified in rats, where a nucleotide substitution in the ND1 genes results in a loss of histocompatibilityl¹²; this peptide is different than the ND1 peptide from mice which is not surprising since different MHC class I proteins preferentially present peptides with different binding motifs. Since mitochondrial peptides bound to class I molecules and displayed at the cell surface can serve as histocompatibility antigens in mice and rats, it is possible that similar systems may be present in other mammalian species.

### DETAILED DESCRIPTION OF THE INVENTION

Thus, the present invention relates to the derivation of desired differentiated cells and tissues from cloned nuclear transfer embryos, wherein such methods generally involve
(i) obtaining a nuclear transfer embryo or parthenogenically activated embryo;
(ii) deriving desired differentiated cells from said embryo or from pluripotent cells derived from said embryo;
(iii) culturing said differentiated cells in vitro or in vivo on a biocompatible matrix device that allows for said cells to assemble into a three-dimensional tissue that morphologically and functionally possesses properties characteristic of endogenous tissue; and
(iv) transplanting said three-dimensional tissue or differentiated cells contained on said biocompatible matrix into a recipient in need of cell or tissue therapy.

With respect to the foregoing methods, nuclear transfer embryos and parthenogenic embryos will be produced by methods which are now known in the art. In general, nuclear transfer cloning involves the transplantation or fusion of a desired cell or DNA or nucleus thereof into a suitable recipient cell, e.g., an oocyte, which is enucleated before or after fusion or transplantation, and which is activated before, during or after cell fusion or transplantation to produce a nuclear transfer embryo that if implanted into a female recipient will yield a viable offspring.

Nuclear transfer methods are now well known and are disclosed in detail in U.S. Pat. Nos. 5,945,577; 6,252,133; 6,525,243; 6,548,571; 6,147,276; 2,215,041; 6,235,970; and 6,235,969; all of which are incorporated by reference in their entirety herein. Such cloning methods can use any differentiated or non-differentiated donor cell which includes all somatic, embryonic and germ cell types. This includes quiescent and non-quiescent cells, i.e., donor cells or nuclei that are in G1, G2, or M cell cycle. Suitable donor cells for nuclear transfer cloning can be obtained directly from animals or tissues, or may be cultured in vitro, and a cell isolated from the cell culture which may be synchronized in a particular cell cycle, e.g., G0.

As noted, the donor cell or nucleus or DNA may also be rendered transgenic prior to use thereof as a nuclear transfer donor cell. Additionally, the recipient cell, e.g., oocyte, may be of the same or different species as the donor cell or DNA or nucleus. Methods for introducing genetic modifications into chromosomal DNA are well known and are disclosed in the patents above-referenced.

Alternatively, embryos may be derived by parthenogenic activation of germ cells, i.e., oocytes or sperm cells, and used to produce pluripotant cells from which differentiated cells may be derived. For example, rabbit and human oocytes have been parthenogenically activated to yield embryos that give rise to differentiated cell types.

After embryos are obtained, these embryos are directly differentiated into desired cell types, or cells derived from said embryos will be used to derive desired differentiated cell types. For example, inner cell mass, morula ES cells, or stem cells derived from an NT or parthenogenic embryo may be induced to differentiate into desired cell types, e.g., by contacting with appropriate growth factors and hormones.

The resultant differentiated cells are then placed on a culture matrix that allows said cells to give rise to a three-dimensional tissue that has the morphology and functional characteristics of endogenous tissue, e.g., renal tissue. In general this will comprise placing cells in contact with a biocompatible matrix that is exposed to nutrients and growth factors to enable tissue generation systems for creating three-dimensional bioengineered tissues are known and are disclosed in numerous published patent applications including U.S. 20030096407 (Creation of Tissue Engineered Female Reproduction Organs); U.S. 20030096406 (“Tissue Engineered Uterus”); U.S. 2002 0160510 (“Renovation and repopulation of decellularised tissues and cadaveric organs by stem cells”); U.S. 20020106743 (“Tissue engineering scaffolds promoting matrix protein production”) U.S. 20020028011 (“Device for engineering a bone equivalent”). All of these published patent applications are incorporate by reference in their entirety.

These systems and matrices generally include biocompatible, biodegradable polymers such as polylactides, polyglycolides, polyester, polycaprolactones, polyanhydrides, polyamides, polyurethanes, polyesteramides, polydioxanones, polyacetals, polyketals, polycarbonates, polyorthoesters, polyphosphoesters, polyphosphazenes, polyhydroxybutyrates, polyhyroxyvolerotes, polyalkylene oxalates, polyalkytene succinates, poly (malic acid), poly (amino acids) and copolymers, terpolymers, or combinations and mixtures thereof. Preferred polymers for bioengineering of tissues are polyglycolic acid (PGA) type polymers.

Additionally, bone equivalents are desirably produced using scaffold materials comprised of destructed natural starch-based polymers (See U.S. 20010021530 published d Sep. 13, 2001).

The matrix and scaffold materials may be partially or fully porous to permit nutrient flow, e.g., on the order of 0.50 to 8000 microns. The scaffold material may be an elastic film, flexible sheet, woven or intertwined fibers, or a three-dimensional structure.

The matrix further may comprise materials that facilitate tissue attachment and generation, e.g., insulin-like growth factor, abscorbic acid, angiotension II, transforming growth factor beta (TGF-beta), and the like.

The matrix containing desired cells on its surface will be placed in contact with suitable biologically active agents including androgen inhibitors, polysaccharides, growth factors, hormones, antiangiogenesis factors, salts, minerals, polypeptides, proteins, amino acids, hormones, interferons, cytokines and antibiotics.

Three-dimensional tissues derived from embroid cells may be obtained in vitro and then implanted into a suitable recipient, or the biocompatible, biodegradable matrix containing cells implanted into a suitable recipient.

The cells that may be cultured on such matrices and used to produce tissue for tissue regeneration, which optionally may be transgenic, include any desired cell or tissue suitable for cell or tissue therapy. Examples include by way of example neural cells, renal cells, pancreatic cells, bone cells, cardiac cells, intestinal cells, stomach cells, tracheal cells, corneal cells, etc.

The resultant tissues and cells may be used to treat conditions including damaged organs, myocardial infarction, seizure disorders, multiple sclerosis, stroke, hypertension, cardiac arrest, ischemia, inflammation, age-related loss of cognitive function, radiation damage, cerebral palsy, neurodegenerative disease, Alzheimer's disease, renal disease, bone injury and bone disease, brain or spinal cord trauma, glaucoma, retinal diseases, retinal trauma, heart-lung bypass, autoimmune diseases such as diabetes, lupus, Graves' disease, and other B and T autoimmune diseases, cancers, tumors, other cell proliferation disorders, burns, cartilage repair, facial dermabrasion, mucousal membranes, neurological structures, (retina, auditory neurons, olfactory neurons, etc.) burn and wound repair of the skin, and for reconstruction of damaged or diseased organs.

As noted, the engineered tissue may be administered in conjunction with other therapies. For example, if the engineered tissue is cardiac tissue, the cells or tissues may be administered in combination with cardiac drugs. Alternatively, if the engineered tissue is to be used to treat cancer it may be administered in combination with an anti-neoplastic or chemotherapeutic agent. These materials may be included on the implanted matrix if so desired. A therapeutically effective amount of the cees or tissues will be administered, typically by injection. For example, cardiac cells or tissues will be injected directly into the damaged heart muscle.

In preferred embodiments, human cells and tissues will be generated by culturing a human blastocyst, inner cell mass, ES, stem, or differentiated cells derived from a human embryo, on a biocompatible matrix that facilitates the generation of the desired tissue. Desirably the tissue will exhibit the morphological and exhibit biological functions of the compounding endogenous tissue, e.g., human renal tissue. As discussed in the examples section herein, this has been accomplished with renal cells, cardiac cells and skeletal cells using cells using cells derived from bovine nuclear transfer embryos. It is anticipated that by similar methods human nuclear transfer embryos may be obtained, used to produce blastocyst stage embryos, and cells derived therefrom used to produce desired tissue types by contacting same with appropriate biocompatible, biodegradable polymeric scaffolds and nutrients. This may be accomplished in vitro, and the resultant tissue transplanted into a recipient or alternatively the matrix containing cells may be implanted at a site in need of tissue transplantation, e.g., a wound, a damaged organ, e.g., damaged heart muscle, pancreas, site of liver trauma, and the like.

As further disclosed herein, it has been demonstrated that allogenic mitrochondrial containing tissues are not rejected by recipients and do not elicit B or T mediated rejection reactions, even after a prolonged time. Also, these tissues exhibit expected in vivo functions. These results suggest that engineered tissues derived by human therapeutic cloning should be well tolerated and efficacious in vivo.

In the present invention, for proof of principle we tested the histocompatibility of nuclear-transfer-generated cells and tissues in a large animal model, the cow (Bos taurus). We find that cloned cardiac and skeletal cell implants were not rejected, and they remained viable after being transplanted back into the nuclear donor animal despite expressing a different mtDNA haplotype. We also demonstrate that nuclear transplantation can be used to generate functional renal structures. It has been estimated that by the year 2010 over 2 million patients will suffer from end-stage renal disease alone, at an aggregate cost of over $1 trillion US dollars during the coming decade¹³.

Owing to its complex structure and function¹⁴, the kidney is one of the most challenging organs to reconstruct in the body. Previous efforts at tissue-engineering the kidney have been directed toward development of an extracorporeal renal support system comprising both biologic and synthetic components¹⁵⁻¹⁷. This approach was first described by Aebischer et al¹⁸⁻¹⁹, and is being focused towards the treatment of acute rather than chronic renal failure. Humes et al¹⁵ have shown that the combination of hernofiltration and a renal-assist device containing tubule cells can replace certain physiologic functions of the kidney when they are connected in an extravascular perfusion circuit in uremic dogs. Heat exchangers, flow and pressure monitors, and multiple pumps are required for optimal functioning of this device²⁰⁻²¹.

Although ex vivo organ substitution therapy would be life-sustaining, there would be obvious benefits for patients if such devices could be implanted long-term without the need for an extracorporeal perfusion circuit or immunosuppressive drugs and/or immune modulatory protocols. While synthetic, selectively permeable barriers can be used ex vivo to separate transplanted cells from the immune system of the body, the implantation of such immunoisolation systems would pose significant difficulties in both the long and short term²²⁻²⁵. Here we demonstrate that it may be feasible to use therapeutic cloning to generate functional immune-compatible renal tissues. Cloned renal cells were successfully expanded in vitro, seeded onto renal units, and implanted back into the nuclear donor organism without immune destruction. The cells organized into glomeruli- and tubule-like structures with the ability to excrete toxic metabolic waste products through a urine-like fluid.

### EXAMPLES

### Example 1

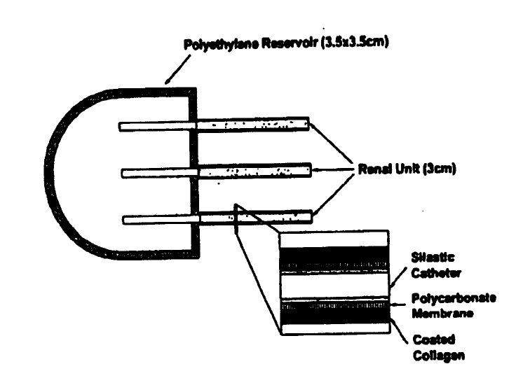

Cardiac and skeletal constructs. Tissue engineered constructs containing bovine cardiac (n=8) and skeletal muscle cells (n=8) were transplanted subcutaneously and retrieved 6 weeks after implantation. After retrieval of the first-set implants, a second set of constructs (n=12) from the same donor were transplanted for a further 12 weeks. On a histological level, the cloned cardiac tissue appeared intact, and showed a well-organized cellular orientation with spindle-shaped nuclei (FIG. 1 A). The retrieved tissue stained positively with troponin I antibodies, indicating the preservation of cardiac muscle phenotype (FIG. 1 B). The cloned skeletal cell explants showed spatially oriented tissue bundles with elongated multinuclear muscle fibers (FIG. 1 D,G). Immunohistochemical analysis using sarcomeric tropomyosin antibodies identified skeletal muscle fibers within the implanted constructs (FIG. 1 F). In contrast to the cloned implants, the allogeneic, control cell implants failed to form muscle bundles, and showed an increased number of inflammatory cells, fibrosis, and necrotic debris consistent with acute rejection (FIG. 1H,1).

Histological examination revealed extensive vascularization throughout the implants, as well as the presence of multinucleated giant cells surrounding the remaining polymer fibers. Although non-degraded fibers were present in all tissue specimens, histomorphometric analysis of the explanted tissues indicated that the degree of immune reaction was significantly less in the cloned versus control tissue sections (66±4 and 54±4 [mean±s.e.m.] total inflammatory cells/HPF/cloned constructs at 6 weeks [first-set grafts] and 12 weeks [second-set grafts], respectively, vs. 93±3 and 80±3 cells/HPF for the constructs generated from the control cells, P<0.0005) (FIG. 1 F-G). Immunocytochemical analysis using CD4- and CD8-specific antibodies identified an approximately twofold increase in CD4+ and CD8+ T cells (13±1.3 and 14±1.4 cells/HPF, respectively, vs. 7±1.1 and 7±1.2 cells/HPF, P<0.00001) within the explanted first and second set control vs. cloned constructs. Importantly, first and second set cloned constructs exhibited comparable levels of CD4 and CD8 expression, arguing against the presence of an enhanced second set reaction as would be expected if mtDNA-encoded minor antigen differences were present.

Polyglycolic acid (PGA) is one of the most widely used synthetic polymers in tissue engineering^{26,27}. PGA polymers are attractive due to their biodegradability and biocompatibility, and have been used in experimental and clinical settings for decades. Although the scaffolds are immune acceptable, the PGA construct is known to stimulate a characteristic pattern of inflammation and in growth similar to that observed in the cloned constructs of the present study. However, this response, which is greatest at around 12 weeks of implantation, can be considered separate from the immune response to the transplanted cells, even though there obviously can be interactions between the two²⁸⁻³³.

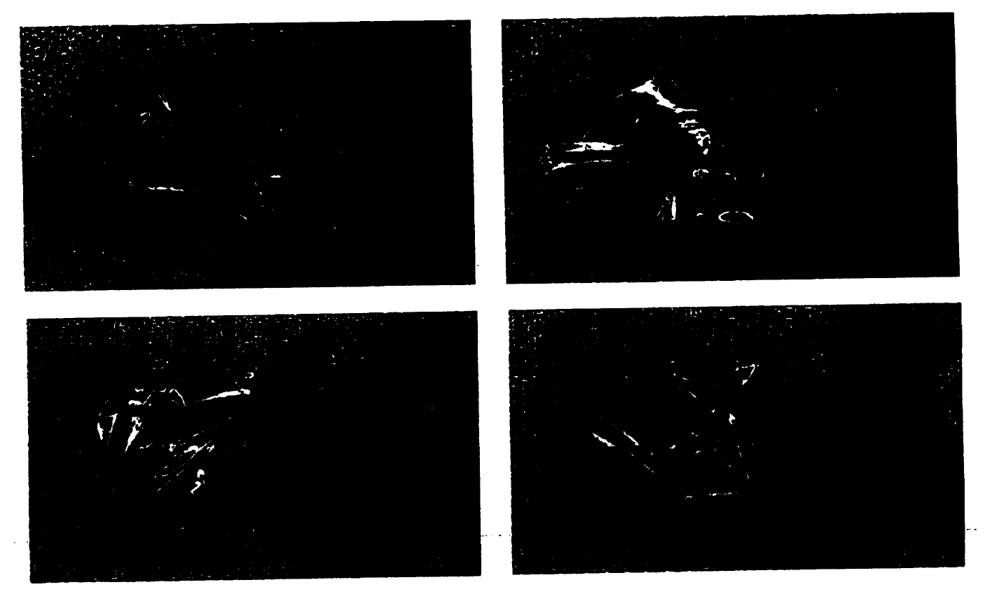

Semi-quantitative RT-PCR and Western blot analysis confirmed the expression of specific mRNA and proteins in the retrieved tissues despite the presence of allogeneic mitochondria. Mean expression intensities of myosin/GAPDH and troponin T/GAPDH in the cloned skeletal and cardiac implants were 0.22±0.03 and 0.15±0.02 (6 weeks) and 0.09±0.08 and 0.29±0.1 (12 weeks), respectively. In contrast, expression intensities were significantly lower or absent in constructs generated from genetically unrelated cattle (0.02±0.01 and 0±0.00 at 6 weeks, P<0.005; and 0±0.01 and 0.02±0.1 at 12 weeks, P<0.05)(FIG. 2A,B). The cardiac and skeletal explants also expressed high protein levels of desmin and troponin I as determined by Western blot analysis (FIG. 2C,D). Desmin expression was significantly greater in the cloned versus control tissue sections (85±1 and 68±4 vs. 30±2 and 16±2 at 6 weeks for the skeletal and cardiac implants, respectively, P<0.001; and 80±3 and 121±24 vs. 53±2 and 52±8 at 12 weeks for the constructs generated from the skeletal and cardiac cells, P<0.05). The expression intensities of troponin I in the cloned and control cardiac muscle explants was 68±4 and 16±2 at 6 weeks (P<0.001), respectively, and 94±7 and 54±12 at 12 weeks (P<0.05).

Western blot analysis of the first-set explants indicated an approximately six-fold increase in expression intensity of CD4 in the control versus cloned constructs at 6 weeks (30±10 and 32±3 for the control skeletal and cardiac implants, respectively, vs. 5±1 and 5±1 for the cloned skeletal and cardiac constructs)(P<O. 0005), confirming a primary immune response to the control grafts. There was also a significant increase in the mean expression intensities of CD8 in the control versus cloned constructs at 6 weeks (26±5 vs. 15±4, P<0.05). Twelve weeks after second-set implantation, mean expression intensities of CD4 and CD8 continued to remain significantly elevated in the control vs cloned constructs (23±4 vs. 12±3 for CD4, respectively, and 54±7 vs. 26±2 for CD8, P<0.005).

### Example 2

Renal constructs. Renal cells were isolated from a 56-day-old cloned fetus and passaged until the desired number of cells were obtained. In vitro immunocytochernistry confirmed expression of renal specific proteins, including synaptopodin (produced by podocytes), aquaporin 1 (AQPI, produced by proximal tubules and the descending limb of the loop of Henle), aquaporin 2 (AQP2, produced by collecting ducts), Tamm-Horsfall protein (produced by the ascending limb of the loop of Henle), and factor VIII (produced by endothelial cells). Synaptopodin and AQP1 & 2 expressing cells exhibited circular and linear patterns in two-dimensional culture, respectively. After expansion, the renal cells were shown to produce both erythropoietin and 1,25-dihydroxyvitamin D₃, a key endocrinologic metabolite. The cloned cells produced 2.9±0.03 mlU/ml of erythropoietin (compared to 0.0±0.03 for control fibroblasts [P<0.0005] and 2.9±0.39 mlU/ml for control renal cells) and were responsive to hypoxic stimulation (5.4±1.01 mlLl/ml at 1% O₂ VS 2.9±0.03 mlU/ml at 20% O₂; P<0.02); 1,25-dihydroxyvitamin D₃ levels were 20.2±1.12 pg/ml, compared to <1 pg/ml for control fibroblasts [P<0.0002] and 18.6±1.72 pg/ml for control renal cells.

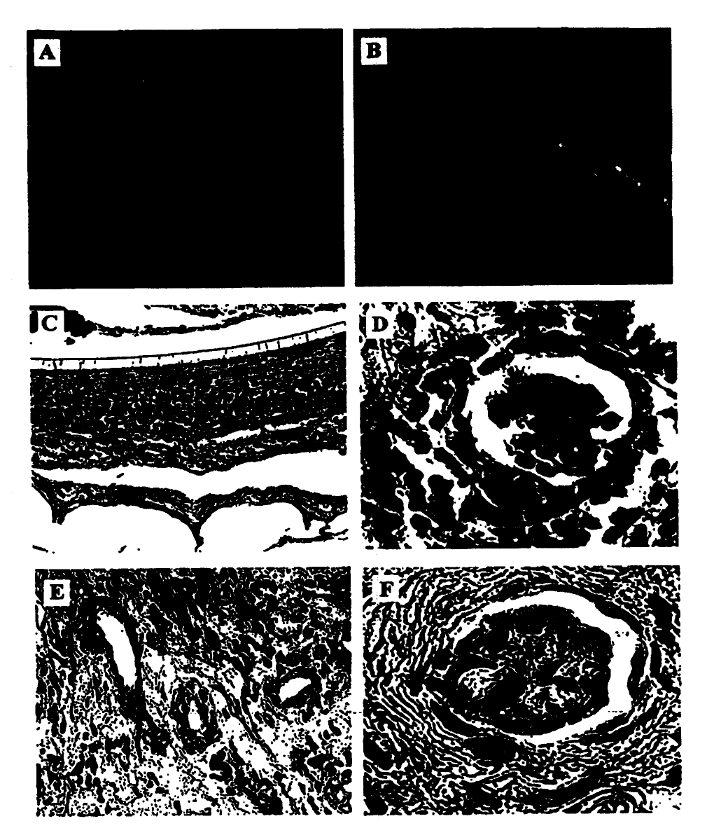

After expansion and characterization, the cloned cells were seeded onto collagen-coated cylindrical polycarbonate membranes. Renal devices with collecting systems were constructed by connecting the ends of three membranes with catheters that terminated in a reservoir (FIG. 3A). Thirty-one units (n=19 with cloned cells, n=6 without cells, and n=6 with cells from an allogeneic control fetus) were transplanted subcutaneously and retrieved 12 weeks after implantation back into the nuclear donor animal.

On gross examination, the explanted units appeared intact, and straw-yellow colored fluid could be observed in the reservoirs of the cloned group (FIG. 3D). There was a six-fold increase in volume in the experimental group vs the control groups (0.60±0.04 ml vs 0.10±0.01 ml and 0.13±0.04 ml in the allogeneic and unseeded control groups, respectively, P<0.00001). Chemical analysis of the fluid suggested unidirectional secretion and concentration of urea nitrogen (18.3±1.8 mg/dl urea nitrogen in the cloned group vs 5.6±0.3 mg/dl and 5.0±0.01 mg/di in the allogeneic and unseeded control groups, respectively, P<0.0005) and creatinine (2.5±0.18 mg/dl creatinine in the cloned group vs 0.4±0.18 mg/dl and 0.4±0.08 mg/dl in the allogeneic and unseeded control groups, respectively, P<0.0005). Although the ratios of urine to plasma urea and creatinine were not physiologically normal, they were significantly increased compared to controls, approaching up to 60% of what is considered within normal limits (i.e. urine to plasma creatinine ratio of 6:1 in the cloned constructs vs. 10:1 in normal kidneys).

Physiological function of the implanted units was further evidenced by analysis of the electrolyte levels in the collected fluid as well as specific gravity and glucose concentrations. The electrolyte levels detected in the fluid of the experimental group were significantly different from plasma or the controls (see Table 1). These findings indicate that the implanted renal cells possess filtration, reabsorption and secretory functions. Urine specific gravity is an indicator of kidney function and reflects the action of the tubules and collecting ducts on the glomerular filtrate by furnishing an estimate of the number of particles dissolved in the urine. The urine-specific gravity of cattle is reported as approximately 1.025 (vs 1.027±0.001 for the fluid that was produced by the cloned renal units), and normally ranges from 1.020 to 1.040 (vs approximately 1.010 in normal bovine serum)^{34,35}. The normal range of urine pH for adult herbivores is alkaline, with values ranging from 7.0 to 9.035 (the pH of the fluid from the cloned renal units was 8.1±0.20). Glucose is reabsorbed in the proximal tubules, and is seldom present in the urine of cattle. Glucose was undetectable (<10 mg/dL) in the cloned renal fluid (vs blood glucose concentrations of 76.6±0.04 mg/dL). The rate of excretion of minerals in cattle depends on a number of variables including their concentration in the animals feed³⁴. However, magnesium and calcium, which are both reabsorbed in the proximal tubules and loop of henle, are normally<2.5 mg/dL and <5 mg/dL in bovine urine, respectively, and were 0.9±0.52 mg/dL and 4.9±1.5 mg/dL in the cloned urine-like fluid, respectively.

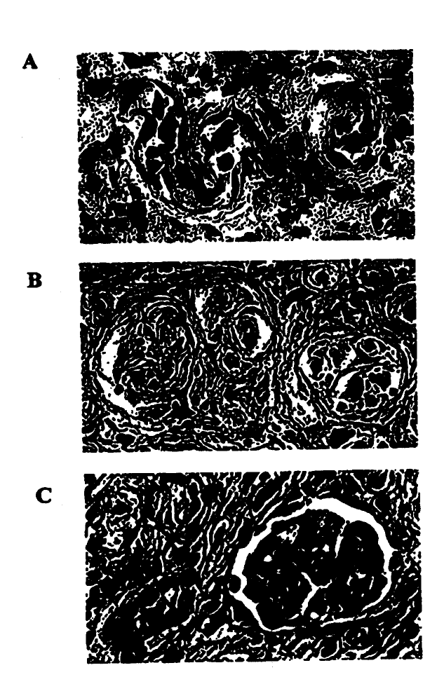

The retrieved implants demonstrated extensive vascularization, and had self-assembled into glomeruli and tubule-like structures (FIG. 4). The latter were lined with cuboid epithelial cells with large, spherical and pale-stained nuclei, whereas the glomeruli structures exhibited a variety of cell types with abundant red blood cells. There was a clear continuity between the mature glomeruli, their tubules, and the polycarbonate membrane (FIG. 4G). The renal tissues were integrally connected in a unidirectional manner to the reservoirs, resulting in the excretion of dilute urine into the collecting systems.

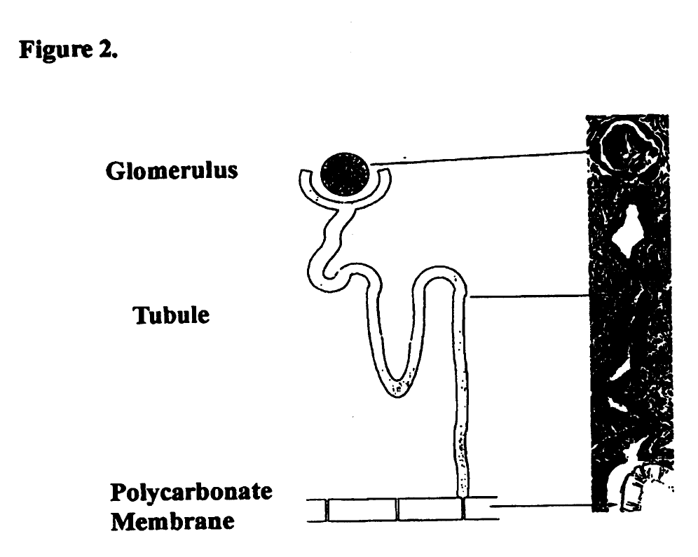

Immunohistochemical analysis confirmed expression of renal specific proteins, including AQP1, AQP2, synaptopodin, and factor VIII (FIG. 5). Antibodies for AQP1, AQP2, and synaptopdin identified tubular, collecting tubule, and glomerular segments within the constructs, respectively. In contrast, the allogeneic controls displayed a foreign body reaction with necrosis, consistent with the finding of acute rejection. RT-PCR analysis confirmed the transcription of AQP1, AQP2, synaptopodin, and Tamm-Horsfall genes exclusively in the cloned group (FIG. 5). Cultured and cloned cells also expressed high protein levels of AQP1, AQP2, synaptopodin, and Tamm-Horsfall protein as determined by Western blot analysis. Expression intensity of CD4 and CD8, markers for inflammation and rejection, were also significantly higher in the control vs cloned group (FIG. 5).

### Example 3

Mitochondrial DNA (mtDNA) analysis. Previous studies showed that bovine clones harbor the oocyte mtDNA^{6-8,36}. As discussed above, differences in mtDNA-encoded proteins expressed by clone cells could stimulate a T cell response specific for mtDNA-encoded minor histocompatibility antigens (miHA)³⁷ when clone cells are transplanted back to the original nuclear donor. The most straight-forward approach to resolve the question of miHA involvement is the identification of potential antigens by nucleotide sequencing of the mtDNA genomes of the clone and fibroblast nuclear donor. The contiguous segments of mtDNA that encode 13 mitochondrial proteins and tRNA's were amplified by PCR from total cell DNA in five overlapping segments. These amplicons were directly sequenced on one strand with a panel of sequencing primers spaced at 500 bp intervals.

The resulting nucleotide sequences (13,210 bp) revealed nine nucleotide substitutions (Table 2) for the first donor:recipient combination (cardiac/skeletal constructs). One substitution was in the tRNA-Gly segment and five substitutions were synonymous. The sixth substitution, in the ND1 gene, was heteroplasmic in the nuclear donor where one of the two alternative nucleotides was shared with the clone. A Leu or Arg would be translated at this position in ND1. The eighth and ninth substitutions resulted in amino acid (M) interchanges of Asn>Ser and Val>Ala in the ATPase6 and ND4L genes, respectively. For the second donor:recipient combination (renal constructs), we obtained 12,785 bp from both the clone and nuclear donor animal. The resulting sequences revealed six nucleotide substitutions (Table 2). One substitution was in the tRNA-Arg segment and three substitutions were synonymous. The fifth and sixth substitutions resulted in AA interchanges of Ile>Thr and Thr>Ile in the ND2 and ND5 genes, respectively. The identification of two AA substitutions that distinguish the clone and the nuclear donor confirm that a maximum of only two miHA peptides could be defined by the second donor:recipient combination. Given the lack of knowledge concerning peptide binding motifs for bovine MHC class I molecules, there is no reliable method to predict the impact of these AA substitutions on the ability of mtDNA-encoded peptides to either bind to bovine class I molecules or activate CD8+ CTLs

Despite the potential involvement of this minimal number of AA substitutions, it was clear that the clone devices functionally survived for the duration of these experiments without significant increases in infiltration of second-set devices by CD4+ and CD8+ T lymphocytes. Specifically, cloned cardiac and skeletal tissues remained viable >3 months after second-set transplantation (comparable to in vitro control specimens). Multiple, viable, myosin- and troponin 1-containing cells were observed throughout the tissue constructs, consistent with functionally active protein synthesis and expression. This direct and relevant assessment of graft function does not provide any evidence to support the activation of a T cell response to cloned tissue-specific histocompatibility antigens in this donor:recipient combination.

These findings are consistent with those observed for the second transplant donor:recipient combination. Although the cloned renal cells derived their nuclear genome from the original fibroblast donor, their mtDNA was derived from the original recipient oocyte. A relatively limited number of mtDNA polymorphisms have been shown to define maternally transmitted miHA in mice³⁸. This class of miHA has been shown to stimulate both skin allograft rejection in vivo and expansion of cytotoxic T lymphocytes (CTL) in vitro³⁸, and could constitute a barrier to successful clinical use of such cloned devices as hypothesized for chronic rejection of MHC-matched human renal transplants^{39,40}. We chose to investigate a possible anti-miHA T cell response to the cloned renal devices through both delayed-type hypersensitivity (DTH) testing in vivo and Elispot analysis of IFNg-secreting T cells in vitro. An in vivo assay of anti-miHA immunity was chosen based on the ability skin allograft rejection to detect a wide range of miHA in mice with survival times exceeding 10 weeks⁴¹ and the relative insensitivity of in vitro assays in detecting miHA incompatibility, highlighted by the requirement for in vivo priming to generate CTL⁴². We were unable to discern an immunological response directed against the cloned cells by DTH testing in vivo. Cloned and control allogeneic cells were intra-dermally injected back into the nuclear donor animal 80 days after the initial transplantation. A positive DTH response was observed after 48 hours for the allogeneic control cells but not the cloned cells (diameter of erythema/induration approx 9×4.5 mm, 12×10 mm, and 11×11 mm vs 0, 0, and 0 mm, respectively, P<0.02).

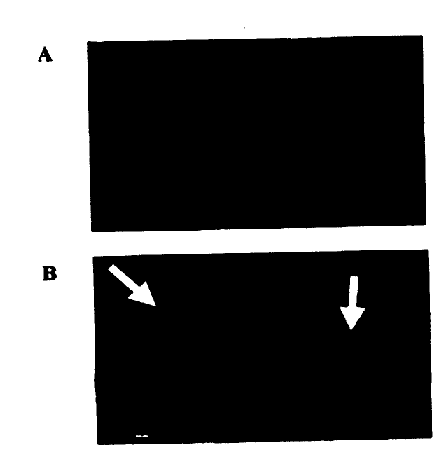

The results of DTH analysis were mirrored by Elispot-derived estimates of the frequencies of T cells that secreted IFN-gamma following in vitro stimulation. PBLs were harvested from the transplanted recipient 1 month after retrieval of the devices. These PBLs were stimulated in primary mixed lymphocyte cultures (MLCs) with allogeneic renal cells, cloned renal cells, and nuclear donor fibroblasts. Surviving T cells were re-stimulated in anti-IFN-gamma-coated wells with either nuclear donor fibroblasts (autologous control) or the respective stimulators used in the primary MLCs. Elispot analysis revealed a relatively strong T cell response to allogeneic renal stimulator cells relative to the responses to either cloned renal cells or nuclear donor fibroblasts (FIG. 6). A mean of 342 spots (s.e.±36.7) was calculated for allogeneic renal cell-specific T cells. Significantly lower numbers of IFN-gamma-secreting T cells responded to cloned renal cells and nuclear donor fibroblasts. Nuclear donor fibroblast-stimulated T cells yielded 45 (s.e.±1.4) and 55 (s.e.±5.7) spots following secondary stimulation with cloned renal and nuclear donor fibroblast stimulators, respectively. Likewise, cloned renal cell-stimulated T cells yielded 61 (s.e.±2.8) and 33.5 (s.e.±0.7) spots with those same stimulator populations. These results corroborate both the relative CD4 and CD8 expression in Western blots (FIG. 5) as well as the results of in vivo DTH testing to support the conclusion that there was no detectable rejection response that was specific for cloned renal cells following either primary or secondary challenge.

Our results suggest that cloned cells and tissues can be grafted back into the nuclear donor organism without immune destruction despite having allogeneic mtDNA, although further studies will be necessary to rule out the possibility of immune rejection with other donor: recipient transplant combinations. Related to the invention, human and primate ES cells have been successfully differentiated in vitro into derivatives of all three germ layers, including beating cardiac muscle cells, smooth muscle, and insulin-producing cells, among others⁴³⁻⁴⁸. In humans, however, there is an ethical consensus not to allow preimplantation embryos to develop in vitro beyond the blastocyst stage; but rather to derive primordial stem cells from the inner cell mass as a source of genetically matched cells for transplantation⁴⁹⁻⁵¹. Although functional tissues can be engineered using adult native cells⁵²⁻⁵³, the ability to bioengineer primordial stem cells into more complex functional structures such as kidneys would overcome the two major problems in transplantation medicine: immune rejection and organ shortage. It is clear that a staged developmental strategy will be required to achieve this ultimate goal. The results presented here suggest it is possible to use nuclear transplantation to eliminate the first of these hurdles, namely, the problem of immune incompatibility.

### Materials and Methods Used in Examples

Experimental Protocol

Adult bovine cell line derivation. Dermal fibroblasts were isolated from adult Holstein steers by ear notch. The tissue sample was minced and cultured in DMEM (Gibco, Grand Island, N.Y.) supplemented with 15% fetal calf serum (HyClone, Logan, Utah), L-glutamine (2 mM), non-essential AA (100 μM), β mercaptoethanol (154 μM) and antibiotics at 38° C. in a humidified atmosphere of 5% CO₂ and 95% air. The tissue explants were maintained in culture and a fibroblast cell monolayer established. The cell strain was maintained in culture, passaged and cryopreserved in 10% DMSO and stored in liquid nitrogen prior to nuclear transfer. Experimental protocols followed guidelines approved by the Children's Hospital and ACT Institution Animal Care and Use Committees Nuclear transfer and embryo culture. Bovine oocytes were obtained from abattoir-derived ovaries as previously described³⁰ Oocytes were mechanically enucleated at 18-22 h postmaturation, and complete enucleation of the metaphase plate confirmed with bisBenzimide (Hoechst 33342; Sigma, St. Louis, Mo.) dye under fluorescence microscopy. A suspension of actively dividing cells was prepared immediately prior to nuclear transfer. Single donor cells were selected and transferred into the perivitelline space of the enucleated oocytes. Fusion of the cell-oocyte complexes was accomplished by applying a single pulse of 2.4 kV/cm for 15 μs. Nuclear transfer embryos were activated as previously described by Presicce et al⁴⁶ with slight modifications. Briefly, reconstructed embryos were exposed to 5 μM of Ionomycin (CalBiochem, La Jolla, Calif.) in TL Hepes for 5 min at RT followed by a 6 h incubation with 5 μg/ml of Cytochalasin B (Sigma) and 10 μg/ml of Cycloheximide (Sigma) in ACM media. Resulting blastocysts were non-surgically transferred into progestrin-synchronized recipients.

Cell culture and seeding. Cardiac and skeletal tissue from 5-6 week-old cloned and natural fetuses were retrieved. The cells were isolated by the explant technique and cultured using DMEM as above. Both muscle cell types were expanded separately until desired cell numbers were obtained. The cells were trypsinized, washed and seeded in 1×2 cm PGA polymer scaffolds with 5×10⁷ cells. Vials of frozen donor cells were thawed and passaged prior to seeding the second-set scaffolds. Renal cells were derived from 7 to 8 week-old cloned and natural fetuses. Metanephros were surgically dissected under a microscope, and cells were isolated by enzymatic digestion using 0.1% collagenase/dispase (Roche, Indianaplois, Ind.), and cultured using DMEM supplemented as above. Cells were passed by 1:3 or 1:4 every 3 to 4 days, and expanded until desired cell numbers (approximately 6×10⁸) were obtained. The cells were seeded in coated collagen with 2×10⁷ cells/cm² density. Vials of frozen donor cells were thawed and passaged for DTH testing and for use in the vitro proliferative assays.

Polymers and renal devices. Unwoven sheets of polyglycolic acid polymers (1 cm×2 cm×3 mm) were used as cell delivery vehicles (Albany International, Mansfield, Mass.). The polymer meshes were composed of fibers of 15 μm in diameter and an interfiber distance between 0-200 um with 95% porosity. The scaffold was designed to degrade via hydrolysis in 8-12 weeks. Renal devices with collecting systems were constructed by connecting the ends of three cylindrical polycarbonate membranes (3 cm long, 10 μm thick, 2 μm pore size, 1.4 mm I.D.; Nucleopore Filtration Products, Cambridge, Mass.) with 16 G Silastic catheters that terminated in a 2 ml reservoir made from polyethylene sealed along the edge by the application of pressure and heat. The superior aspect of the cylindrical membranes was also sealed, and the membranes coated with type 1 collagen (0.2 cm thickness) extracted from rat-tail collagen prior to use.

Implantation and analysis of fluid. The cell-polymer constructs were implanted into the flank subcutaneous tissue of the same steer from which the cells were cloned. Fourteen constructs (8 first-set and 6 second-set) for each cell type were implanted. Control group constructs, with cells isolated from an allogeneic fetus, were implanted on the contralateral side. The implanted constructs were retrieved at 6 weeks (first-set) and 12 weeks (second-set) after implantation. The renal units were also derived from a single fetus. Thirty-one units (n=19 with cloned cells, n=6 without cells, and n=6 with cells isolated from an allogeneic, age-matched control fetus) were transplanted subcutaneously and retrieved 12 weeks after implantation. The solute concentrations of urea nitrogen, creatinine, and electrolytes were measured in the accumulated fluid in the explanted renal reservoirs using standard techniques.

DTH testing. Cloned, allogeneic and autologous cells were intra-dermally injected into the nuclear donor animal (11×10⁶ cells in 0.1 ml in triplicate). Three sites were chosen with the softest skin: the left and right side of the tail, and just below the anus. After each site was shaved and prep'd, the cells were injected in a row about 2 cm apart. The area of erythema and induration was measured (blinded) after 24-72 hours, with 48 hours being considered the optimal time to detect a DTH response.

Elispot. Bovine recipient PBLs were isolated from whole blood and cultured for six days with irradiated allogeneic renal cells, cloned renal cells, and nuclear donor fibroblasts at 37° C. in RPMI plus 10% FCS and human IL-2 (20 U/ml). On day six, the stimulated PBLs were harvested and plated at 25,000 cells/well in duplicate wells of a 96 well Multiscreen plate, which had been coated overnight with mouse anti-bovine IFN gamma (10 μg/ml) (Biosource, Camarillo, Calif.). Fifty-thousand cells matched to the primary culture stimulators were added to the respective wells. The plate was incubated for 24 hr at 37° C. and washed 3× with 0.5% Tween-20 and 4× in distilled water. Biotinylated mouse anti-bovine IFN-gamma (5 μg/ml) (Biosource) was added, and the plate was incubated for 2 hours at 37° C. The plate was washed as above and alkaline phosphatase-conjugated anti-biotin (1/1000 dilution) (Vector, Burlingame, Calif.) was added and incubated for 1 hour at RT. The plate was washed and 100 μl of BCIP/NBT (Sigma) was added for development of spots. After development, BCIP/NBT was washed out of the wells with distilled water. The wells were photographed and analyzed with Immunospot software (Cellular Technologies, Cleveland, Ohio).

Histological and immunohistochernical analyses. Five-micron sections of 10% buffered formalin fixed paraffin-em bedded tissue were cut and stained with H&E. Immunohistochemical analyses were performed using specific antibodies in order to identify the cell types in retrieved tissues with cryostat and paraffin sections. Monoclonal sarcomeric tropomyosin (Sigma) and troponin I (Chemicon, Temecula, Calif.) antibodies were used to detect skeletal and cardiac fibers, respectively. Monoclonal synaptopodin (Research Diagnostics Inc, Flanders, N.J.), polyclonal AQP1, AQP2 and polyclonal Tamm-Horsfall protein (Biomedical Technologies Inc, Stoughton, Mass.) were used to detect glomerular and tubular tissue, respectively. Monoclonal CD4 and CD8 (Serotec, Raleigh, N.C.) antibodies were used to identify T cells for immune rejection. Specimens were routinely processed for immunostaining. Pretreatment for high-temperature antigen unmasking pretreatment with 0.1% trypsin was performed using a commercially available kit according to the manufacturers recommendations (T-8128; Sigma). Antigen-specific primary antibodies were applied to the deparaffinized and hydrated tissue sections. Negative controls were treated with nonimmune serum instead of the primary antibody. Positive controls consisted of normal tissue. After washing with phosphate buffered saline, the tissue sections were incubated with a biotinylated secondary antibody and washed again. A peroxidase reagent (DAB) was added. Upon substrate addition, the sites of antibody deposition were visualized by a brown precipitate. Counterstaining was performed with Gill's hematoxylin. For determining the degree of immunoreaction, the immune cells were counted under 5 high power fields per section (HPF, ×200) using computerized histomorphometrics (Biolmaging Analyses Software).

Erythropoietin and 1,25-dihydroxyvitamin D3assays. Cloned renal cells, allogeneic renal cells, and cloned fibroblasts were grown to confluence in 60 mm culture dishes (in quantruplicate) at 20% O₂, 5% CO₂. After washing 3× the cells were incubated in either serum-free medium for 24 hours (erythropoietin) or serum-free medium with 25-hydroxyvitamin D₃ (1 ng/ml) for 12 hours (1,25-D₃). Erythropoietin production in the supernatants was measured by the double-antibody sandwich enzyme-linked immunosorbent assay using a Quantikine® IVD® Erythropoietin ELISA kit (R&D Systems, Minneapolis, Minn.). Erythropoietin production was also measured in the supernatant of cells that were incubated in a hypoxic chamber (1% O₂, 5% CO₂) for 4 hours. 1, 25-dihydroxyvitamin D₃ production in the supernatants was measured by radioimmunoassay using a ¹²⁵I RIA kit (DiaSorin Inc., Stillwater, Minn.).

Mitochondrial DNA analyses. Mitochondrial DNA products ranging in size from 3-3.8 kb were amplified by PCRs using Advantage-GC Genomic Polymerase (Clontech, Palo Alto, CA) and total genomic DNA templates from the clone and nuclear donor. The regions of the mitochondria that were amplified included all of the protein-coding sequences and the intervening tRNAs. PCR products were electrophoresed in 1% SeaPlaque GTG agarose (Rockland, Me.), extracted from the gels with the use of QIAquick Gel Extraction Kits (Qiagen, Valencia, Calif.), and sequenced by the Molecular Biology Core Facility (Mayo Clinic) with a series of primers located approximately 500 base intervals.

RNA isolation, cDNA synthesis. Fresh retrieved tissue implants were harvested and frozen immediately in liquid nitrogen. The tissue was homogenized in RNAzol reagent at 4° C. using a tissue homogenizer. RNA was isolated according to the manufacturers protocol (Tel-Test). Complementary DNA was synthesized from 2 ug RNA using the SuperscriptII reverse transcriptase (Gibco) and random hexamers as primers. PCR. For PCR amplification 1 ml of cDNA with 1 U Taq DNA polymerase (Roche), 200 mM dNTP and 10 pM of each primer were used in a final volume of 30 ml. Myosin for skeletal muscle tissue was amplified from cDNA with primers 5′-TGAATTCMGGAGGCGTTTCT-3′ and 5′-CAGGGCTTCCACTTCTTCTTC-3′. Troponin T for cardiac tissue was done with primer 5′-MGCGCATGGAGMGGACCTC-3′ and 5′-GGATGTAGCCGCCGAAGTG-3′. Synaptopodin for glomerulus was amplified from cDNA with primers 5′-GGTGGCCAGTGAGGAGGM-3′ and 5′-TGCTCGCCCAGACATCTCTT-3′. Podocalyxin for glomerulus was done with primer 5′-CTCTCGGCGCTGCTGCTACT-3′ and 5′CGCTGCTGGTCCTTCCTCTG-3′. AQP1 for tubule was done with primer 5′-CAGCATGGCCAGCGACGAGTTCMGA-3′ and 5′-TGTCGTCGGCATCCAGGTCATAC-3′, AQP2 for tubule was done with primer 5′-GCAGCATGTGGGARCTNM-3′ and 5′-CTYACIGCRTTIACNGCNAGRTC-3′. Tamm-Horsfall protein for tubule was done with primer 5′-AACTGCTCCGCCACCM-3′ and 5′-CTCACAGTGCCTTCCGTCTC-3′. PCR products were visualized With agarose gel electrophoresis and ethidiurn bromide staining.

Western blot analysis. Tissue was homogenized in lysis buffer using a tissue homogenizer. After measuring protein concentration (BioRad), equal protein amounts were loaded on 10% SDS-PAGE. Proteins were blotted onto PVDF-membranes, the membranes were incubated with primary antibodies for 1 h at RT. Desmin (Santa Cruz Biotech, Santa Cruz, Calif.) antibodies were used to detect skeletal tissue; desmin (Santa Cruz Biotech) and troponin I antibodies were used to detect cardiac tissue; and synaptopodin (Research Diagnostics inc., Flanders, N.J.), AQP1, AQP2, and Tamm-Horsfall protein were used to detect glomerular and tubular tissue, respectively. Monoclonal CD4 and CD8 antibodies were used as markers for inflammation and rejection. Subsequently membranes were incubated with secondary antibodies for 30 minutes. The signal was visualized using the ECL system (NEN, Boston, Mass.).

Statistical analysis. Data are presented as mean±s.e.m. and compared using the two-tailed Student's t test. Differences were considered significant at P<0.05.

TABLE 1
Chemical analysis of fluid produced by renal units

| | Blood | Control 1 | Control 2 | Cloned |
|---|---|---|---|---|
| Sodium (mmol/L) | 141.7 ± 0.66* | 140.7 ± 0.67* | 141.3 ± 0.67* | 133.2 ± 2.10* |
| Potassium (mmol/L) | 4.5 ± 0.03* | 7.4 ± 0.28 | 7.5 ± 0.63 | 9.3 ± 0.34* |
| Chloride (mmol/L) | 97.7 ± 1.33* | 105.3 ± 0.33* | 105.5 ± 0.21 | 79.3 ± 7.53* |
| Calcium (mg/dL) | 10.2 ± 0.06* | 6.6 ± 0.17 | 6.5 ± 0.33 | 4.9 ± 1.50* |
| Magnesium (mg/dL) | 2.6 ± 0.03* | 2.4 ± 0.05* | 2.5 ± 0.12* | 0.9 ± 0.52* |

Mean ± s.e.m.
*P < 0.05 (comparison between each blood, control and cloned groups in the same conditions)

TABLE 2
Nucleotide and amino acid substitutions that distinguish
the nuclear donor and cloned cells

| | Nuclear | | | Amino Acid |
|---|---|---|---|---|
| Clone | Donor | Position ^{a} | Gene | Substitution |
| First Combination | | | | |
| A | G | 13,080 | ND5 | — |
| T | C | 14,375 | ND6 | — |
| T | C | 7,851 | Coll | — |
| C | T | 8,346 | ATPase6 | — |
| A | G | 8,465 | ATPase6 | N > S |
| G | G/T | 3,501 | ND1 | R? L/R |
| C | T | 9,780 | tRNA-Gly | |
| T | C | 10,432 | ND4L | V? 4A |
| G | A | 11,476 | ND4 | |
| Second combination | | | | |
| T | C | 4,945 | ND2 | I > T |
| C | T | 7,580 | COII | — |
| A | G | 9,095 | COIII | — |
| C | T | 10,232 | tRNA-Arg | — |
| G | A | 10,576 | ND4 | — |
| C | T | 12,377 | ND5 | T > I |

^{a} Position in Genbank #J013494

The results contained in this application support a conclusion that tissue-engineered constructs of different tissue types can be obtained by culturing cells derived from nuclear transfer or parthenogenic embryos, in the presence of a matrix that promotes tissue development. Typically such matrices will comprise a biocompatible polymer such as one known in the art for promoting tissue development. In the present invention, the cells cultured preferably will be produced by nuclear transfer, and include e.g., cultured inner cell masses, morula, ES cells, non-embryonic stem cell types such as hematopoietic stem cells, and differentiated cells derived from nuclear transfer embryos such as kidney cells, cardiac cells, esophageal cells, etc.

However, the invention also embraces the use of cells derived by methods other than nuclear transfer, e.g., ICMs, morulas and blastocysts produced by IVF, ICMs and stem cells derived from embryos produced by parthenogenesis or androgenesis, somatic cells that have been converted into a desired cell type by transfer of cytoplasm from another type of somatic cell (to convert one somatic cell into a different somatic cell lineage), ES and other pluripotent cells produced by cytoplasmic transfer, i.e., by transfer of cytoplasm from oocytes or other embryonic cells), as well as differentiated cells derived from any of the foregoing.

Also, the invention embraces the same types of cells which are transgenic, e.g., by incorporation of a desired heterologous DNA or by deletion of an endogenous DNA. Transgenic cells may be obtained by known methods, e.g., by use of retroviral vectors, microinjection, homologus recombination, etc. Preferably, the transgene will be inserted or deleted at a predetermined site by use of targeted integration or deletion. The tissue-engineering methods disclosed in the invention may be used to provide any desired tissue engineered construct, e.g., lung, liver, bladder, blood vessels, trachea, esophagus, cartilage, skin, bone, muscle, ligaments, tendons, cornea, parethynoid, teeth, inner ear, bladder, intestine, stomach, pancreatic islets, functional cardiac tissue, liver, gall bladder, reproductive tissue, and other tissue types.

### REFERENCES

1 Lanza, R. P. et al. The ethical reasons for stem cell research. Science 293, 1299 (2001).
2. Atala, A. & Lanza, R. P. Methods of tissue engineering (Academic Press, San Diego, Calif.; 2001).
3. Atala, A. & Mooney, D. Synthetic biodegradable polymer scaffolds (Birkhauser, Boston, Mass.; 1997).
4. Machluf, M. & Atala, A. Emerging concepts for tissue and organ transplantation. Graft 1, 31-37 (1998).
5. Lanza, R. P., Cibelli, J. B. & West, M. D. Prospects for the use of nuclear transfer in human transplantation. Nat. Biotechnol. 17, 1171-1174 (1999).
6. Evans, M. J. et al. Mitochondrial DNA genotypes in nuclear transfer-derived cloned sheep. Nat. Genet. 23, 90-93 (1999).
7. Hiendleder, S., Schmutz, S. M., Erhardt, G., Green, R. D. & Plante, Y. Mol. Reprod. Dev. 54, 24-31 (1999).
8. Steinborn, R. et al. Mitochondrial DNA heteroplasmy in cloned cattle produced by fetal and adult cell cloning. Nat. Genet. 25, 255-257 (2000).
9. Vyas, J. M. et al. Biochemical specificity of H-2M3a: Stereospecificity and space-filling requirement at position 1 maintains N-formyl peptide binding. J. Immunol. 149, 3605-3611 (1992).
10. Morse, M. et al. The COI mitochondrial gene encodes a minor histocompatibility antigen presented by H2-M3. J. Immunol. 156, 3301-3307 (1996).
11. Loveland, B., Wang, C. R., Yonekawa, H., Hermel, E. & Lindahl, K. F. Maternally transmitted histocompatibility antigens of mice: a hydrophobic peptide of a mitochondrial encoded protein. Cell 60, 971-980 (1990).
12. Davies, J. D. et al. Generation of T cells with lytic specificity for atypical antigens. I. A mitochondrial antigen in the rat. J. Exp. Med. 173, 823-832 (1991).
13. Lysaght, M. J. Maintenance dialysis population dynamics: current trends and long-term implications. J. Am. Soc. Nephrol. 13, S37-S40 (2002).
14. Amiel, G. E. & Atala, A. Current and future modalities for functional renal replacement. UroL Clin. 26, 235-246 (1999).
15. Humes, H. D., Buffington, D. A., MacKay, S. M., Funke, A. J. & Weitzel, W. F. Replacement of renal function in uremic animals with a tissue-engineered kidney. Nat. Biotechnol. 17, 451-455 (1999).
16. Cieslinski, D. A. & Humes, H. D. Tissue engineering of a bioartificial kidney Biotechnol. Bioeng. 43, 781-791 (1994).
17. MacKay, S. M., Kunke, A. J., Buffington, D. A. & Humes, H. D. Tissue engineering of a bioartificial renal tubule ASA10 J. 44, 179-183 (1998).
18. Aebischer, P., Ip, T. K., Panol, G. & Galletti, P. M. The bioartificial kidney: progress towards an ultrafiltration device with renal epithelial cells processing. Life Support Syst. 5, 159-168 (1987).
19. Ip, T., Aebischer, P. & Galletti, P. M. Cellular control of membrane permeability. Implications for a bioartificial renal tubule. ASA10 Trans. 34, 351-355 (1988)
20. Humes, H. D. Renal replacement devices. In: R. P. Lanza, R. Langer & J. Vacanti, Eds Principles of Tissue Engineering, 2^{nd} Ed (Academic Press, San Diego, 2000). Pp. 645-653.
21. Amiel, A., Yoo, J. & Atala, A. Renal therapy using tissue engineered constructs and gene delivery. W J. Urol. 18, 71-79 (2000).
22. Lanza, R. P., Hayes, J. L. & Chick, W. L. Encapsulated cell technology. Nat. Biotech. 14, 1107-1111 (1996).
23. Kuhtreiber, W. M., Lanza, R. P. & Chick, W. L, Eds. Cell Encapsulation Technology and Therapeutics (Birkhauser, Boston, 1998).
24. Lanza, R. P & Chick, W. L., Eds. Immunoisolation of Pancreatic Islets (R. G. Landes, Austin, 1994).
25. Joki, T., Machluf, M., Atala, A., Zhu, J., Seyfried, N. T., Dunn, I. F., Abe, T., Carroll, R. S. & Black, P. M. Continuous release of endostatin from microencapsulated engineered cells for tumor therapy. Nature Bio. 19, 35-39 (2001).
26. Lanza, R. P, Langer, R. & Vacanti, J. Principles of tissue engineering (Academic Press, San Diego, Calif.; 2000).
27. Atala, A. Future perspectives in reconstructive surgery using tissue engineering. Urol. Clin. 26,157-166 (1999).
28. Santavirta, S. et al. Immune response to polyglycolic acid implants. J. Bone Joint Surg. Br. 72, 597-600 (1990).
29. Paivarinta, U. et al. Intraosseous cellular response to biodegradable fracture fixation screws made of polyglycolide or polylactide. Arch. Orthop. Trauma Surg. 112, 71-74 (1993).
30. Bostman, O. M. & Pihlajamaki, H. K. Adverse tissue reactions to bioabsorbable fixation devices. Clin. Orthop. 371, 216-227 (2000).
31. Ruuskanen, M. et al. Evaluation of self-reinforced polyglycolide membrane implanted in the subcutis of rabbits. Ann. Chir. Gynaecol. 88, 308-12 (1999).
32. Weiler, A., Helling, H. J., Kirch, U., Zirbes, T. K. & Rehm, K. E. Foreign-body fracture fixation: experimental study in sheep. J. Bone Joint Surg. Br. 78, 369-76 (1996).
33. Pariente, J. L., Kim, B. S. & Atala, A. In vitro compatibility assessment of naturally-derived and synthetic biomaterials using normal human urothelial cells. J. of Biomed. Mat. Res. 55, 33-39 (2001).
34. Rosenberger, G. Clinical Examination of Cattle (Verlag Paul Parey, Berlin, 1979) pp. 275-281.
35. Smith, B. P. Large Animal Internal Medicine: Diseases of Horses, Cattle, Sheep and Goats, 2^{nd} Ed (Mosby, St. Louis, 1996) pp. 467-469.
36. Lanza, R. P. et al. Cloning of an endangered species (Bos gaurus) using interspecies nuclear transfer. Cloning 2, 79-90 (2000).
37. Fischer Lindahl, K., Hermel, E., Loveland, B. E., Wang, C. R., Maternally transmitted antigen of mice. Ann. Rev. Immunol. 9:351-372 (1991).
38. Fischer Lindahl, K., Hermel, E., Loveland, B. E. & Wang, C. R. Maternally transmitted antigen of mice. Annual Review of Immunology 9:351-372 (1991).
39. Hadley, G. A., Linders, B. & Mohanakumar, T. Immunogenicity of MHC class I alloantigens expressed on parenchymal cells in the human kidney. Transplantation 54, 537-542 (1992).

40. Yard, B. A. et aL., Analysis of T cell lines from rejecting renal allografts. Kidney Intl. 43,S133-SI38(1993).
41. Bailey, D. W. Genetics of histocompatibility in mice. 1. New loci and congenic lines. Immunogenetics 2, 249-256 (1975).
42. Mohanakumar, T. The role of MHC and non-MHC antigens in allograft immunity (R. G. Landes Company, Austin, Tex., 1994) pp. 1-115.
43. Itskovitz-Eldor, J et al. Differentiation of human embryonic stem cells into embryoid bodies comprising the three embryonic germ layers. Mol. Med 5, 88-95 (2000)
44. Schuldiner, M. Yanuka, O., Itskovitz-Eldor, J, Melton, D. A. & Benvenisty, N. Effects of eight growth factors on the differentiation of cells derived from human embryonic stem cells. Proc. Natl. Acad. Sci USA 97, 11307-11312 (2000)
45. Kaufman, D. S. et al. Directed differentiation of human embryonic stem cells into hematopoeitic colony forming cells. Blood 94 (suppl 1, part 1 of 2), 34a (1999).
46. Reubinoff, B. E. et al. Neural progenitors from human embryonic stem cells. Nat. Biotechnol. 19, 1134-1140(2001).
47. Reubinoff, B. E., Pera, M. F., Fong, C. Y., Trounson, A. & Bongso, A. Embryonic stem cell lines from human blastocysts: somatic differentiation in vitro. Nat. Biotechnol. 18, 399-404 (2000).
48. Cibelli, J. B. et al. Parthenogenetic stem cells in nonhuman primates. Science 295, 819(2002).
49. Lanza, R. P., Cibelli, J. B. & West, M. D. Human therapeutic cloning. Nat. Med. 5, 975-977(1999).
50. Cibelli, J. B. et al. Somatic cell nuclear transfer in humans: pronuclear and early embryonic development. e-biomed: J. Regen. Med. 2, 25-31 (2001).
51. Lanza, R. P. et al. The ethical validity of using nuclear transfer in human transplantation. J. A. M. A. 284, 3175-3179 (2000).
52. Oberpenning, F. O., Meng, J., Yoo, J. & Atala, A. De novo reconstitution of a functional urinary bladder by tissue engineering. Nature Biotech. 17, 149-155 (1999).
53. Kaushal, S., Amiel, G., Guleserian, K. J., Perry, T., Shapira, O. M., Sutherland, F. W., Rabkin, E., Moran, A. M., Schoen, F. J., Atala, A., Soker, S., Bischoff, J. & Mayer, J. E. Circulating endothelial cells for tissue engineering of small diameter vessels. Nature Med. 7, 1035-1040 (2001).
54. Presicce, G. A. & Yang, X. Parthenogenetic Development of Bovine Oocytes Matured In Vitro for 24 Hr and Activated by Ethanol and Cycloheximide. MoL Reprod. Dev. 38, 380-385 (1994).

What is claimed:

1. A method for producing tissue engineered constructs having a desired genetic type comprising the following steps:
(i) contacting an inner cell mass, morula, ES cell, stem cell, or desired differentiated cell with a matrix that facilitates generation of a three-dimensional tissue that is suitable for use in cell therapy; wherein said inner cell mass, morula, stem cell, ES cell or differentiated cell is derived from an embryo, which is produced by same species or cross-species nuclear transfer, parthenogenesis or androgenesis or by cytoplasmic transfer of cytoplasm from an embryonic cell into a somatic cell or by transfer of cytoplasm from a somatic cell into a somatic cell of a different lineage.

2. The method of claim 1 wherein the tissue produced is selected from the group consisting of skin, bone, lung, liver, cartilage, muscle, blood vessels, trachea, esophagus, cartilage, muscle, ligaments, tendons, cornea, parthyroid, teeth, inner ear, bladder, intestine, stomach, pancreatic islets, cardiac tissue, liver, gall bladder, and reproductive tissues.

3. A tissue according to claim 2.

4. The tissue of claim 3 which is transgenic.

5. The tissue of claim 4 which is mammalian.

6. The tissue of claim 5 which is human.

7. The tissue of claim 5 which is rabbit, porcine, ovine, equine, canine, caprine, non-human primate, bear, and dog.

8. The method of claim 1 wherein the tissue is human.

9. A method according to claim 1 which further comprises transplanting said tissue engineered construct or cell containing matrix into a recipient.

10. The method of claim 9 wherein said recipient is human.

11. The method of claim 10 wherein said tissue is selected from bone, neural, intestinal, skin, trachea, cornea, retina, tongue, testis, ovary, larynx, lung, bronchi, intestine, live, gall bladder, and bone marrow.

12. The method of claim 9 wherein transplanting is effected to treat a disease or disorder selected from cancer, burn, trauma, stroke, heart disease, heart attach, diabetes, immune dysfunction, AIDS, liver disease, skin disease, corneal disease or injury, spinal cord injury or disease, multiple sclerosis, reproductive dysfunction, lung disease, and auditory dysfunction.

13. The method of claim 9 wherein the engineered tissue is human heart tissue.

14. The method of claim 9 where in engineered tissue is human renal tissue.

15. The method of claim 9 wherein the engineered tissue is human bone.

16. The method of claim 9 wherein the engineered tissue is human pancreatic tissue.

17. The method of claim 9 wherein the engineered tissue is human corneal tissue.

18. The method of claim 9 wherein the engineered tissue is human lung tissue.

19. The method of claim 9 wherein the engineered tissue in human retinal tissue.

20. The method of claim 9 wherein the engineered tissue is human reproductive organ tissue.
